(19) United States
(12) Patent Application Publication
(10) Pub. No.: US 2022/0323636 A1
(43) Pub. Date: Oct. 13, 2022

(54) Sutures with External Filament Containing A Medicant

(71) Applicant: Ethicon, Inc., Somerville, NJ (US)

(72) Inventors: Howard Scalzo, Somerville, NJ (US); Leo Kriksunov, Ithaca, NY (US); Robert J. Tannhauser, Bridgewater, NJ (US)

(21) Appl. No.: 17/708,570

(22) Filed: Mar. 30, 2022

Related U.S. Application Data
(60) Provisional application No. 63/174,162, filed on Apr. 13, 2021.

(51) Int. Cl. A61L 17/00; A61B 17/06; A61L 17/06; A61L 17/04; A61L 17/14

(52) U.S. Cl. CPC A61L 17/005; A61B 17/06004; A61B 17/06066; A61B 17/06166; A61L 17/06; A61L 17/04; A61L 17/145; A61B 2017/06052; A61B 2017/06176; A61L 2300/104; A61L 2300/206; A61L 2300/202

(57) ABSTRACT

The present invention is directed to suturing systems having a needle, an elongated flexible suture having a connecting end attached to a said needle and an opposing free end and at least one elongated external beneficial filament that is attached to said needle or attached to said suture at the connecting end. The beneficial filament has a smaller cross-sectional area and lower mechanical strength than the suture and contains a medicant.

### FIELD

The field of art to which this invention relates is absorbable or non-absorbable medical devices, such as monofilament sutures or braided multifilament sutures, more specifically surgical sutures having externally attached beneficial filaments containing a releasable medicant.

### BACKGROUND

Surgical sutures and attached surgical needles are well known in the art for use in a variety of conventional surgical procedures. For example, such sutures may be used to approximate tissue about incisions or lacerations in epidermal layers and underlying fascia layers, join blood vessel ends, attach tissue to medical devices such as heart valves, repair body organs, repair connective tissue, etc. Conventional surgical sutures may be made from known biocompatible materials, particularly synthetic and natural biocompatible polymeric materials, which may be non-absorbable or absorbable. Examples of synthetic non-absorbable polymeric materials useful to manufacture non-absorbable sutures include polyesters, polyolefins, polyvinylidene fluorides and polyamides. Further examples of non-absorbable materials are polyethylene, polypropylene, nylon, and similar polymers. Examples of synthetic absorbable polymeric materials useful to manufacture absorbable sutures include polymers and copolymers made from lactones such as the lactides, glycolide, p-dioxanone, epsilon-caprolactone, and trimethylene carbonate. The term absorbable is meant to be a generic term, which may also include implantable devices that bioabsorbable, resorbable, bioresorbable, degradable or biodegradable in the living body or tissue. The term non-absorbable is meant for implantable devices that are permanently installed in the living body or tissue.

Sutures are preferred by surgeons for use in many surgical procedures because of several advantages and properties possessed by such sutures. Absorbable sutures must be capable of providing the desired tensile strength in vivo for a sufficient period of time to allow for effective tissue healing. Wound healing is dependent on the nature of the specific tissue as well as the healing characteristics of the individual undergoing the surgical procedure. For example, poorly vascularized tissue is likely to heal more slowly than highly vascularized tissue; likewise, diabetic patients and the elderly tend to heal more slowly as well. There are thus opportunities to provide suture materials that can match the healing characteristics of a variety of wounds. Any implant, such as a suture, appears as a foreign body to the patient's immune system. In addition, it is known that implantable medical devices, including sutures, may provide a platform for the attachment of bacteria and the subsequent formation of bacterial biofilms.

Surgical sutures are designed to have the requisite physical characteristics to assure desirable and efficacious in vivo behavior. Absorbable sutures need to retain appropriate tensile strength during the required healing period; this is typically characterized as breaking strength retention (BSR). In order to obtain the required design properties, it is necessary to provide absorbable polymers and manufacturing processes that will yield absorbable sutures with the required properties.

Likewise, the retention of mechanical properties, including, e.g. tensile strength and knot strength, post-implantation, is often a very important and critical feature of an absorbable medical device. The device must retain mechanical integrity until the tissue has healed sufficiently. In some bodily tissues, healing occurs more slowly, requiring an extended retention of mechanical integrity. As mentioned earlier, this is often associated with tissue that has poor vascularization. Likewise, there are other situations in which a given patient may be prone to poor healing, e.g., the diabetic patient.

It is known to include certain medicants, such as antimicrobials, in or on sutures or other medical implants such as hernia meshes or the like, for rapid release or which must stay in vivo for extended periods of time, so as to reduce the likelihood of post-surgical infections. However, the presence of such substances incorporated into or onto the polymer material of an implant, can undesirably affect its processibility and/or mechanical integrity. It also can undesirably affect other suture parameters such as flexibility, bendability, handleability by the surgeon, knot strength, knot slide, and similar.

In particular, additions of large amounts of a medicant needed to provide substantial antibacterial properties to a suture, either by incorporation into the suture material itself, or via surface coating of the suture, can undesirably affect the suture properties, as stated above. Further, such approaches to incorporating the medicants can be difficult to apply, process, sterilize, and package.

U.S. Pat. No. 8,097,005 “Suture system” discloses a method for implanting a prosthetic device in a body comprising: placing a first suture strand through tissue at a first position using a first needle of a suture system having more than three needles connected by suture strands; placing a second needle of the suture system in the tissue at a distance from the first position, the second needle being attached to the first suture strand and a second suture strand having different indicators such that the first strand and the second strand can be identified, the second needle having a needle diameter at least as large as the diameter of the first suture strand added to the diameter of the second suture strand; placing additional sutures using additional needles attached to the second needle through the tissue; using more than three needles to insert the suture strands through the prosthetic device; and using the suture strands having the indicators to secure the prosthetic device into position.

Patent Publication No. CN210204810U “Double-strand surgical suture needle” discloses a double-strand surgical suture needle that comprises a double-strand phoenix tail thread and a surgical needle; the double-strand phoenix tail thread is composed of three double-strand absorbable sutures and a joint; the side of the joint is provided with a circle of semicircular grooves, and the left end of the joint; Three double-strand absorbable sutures are fixedly connected; the surgical suture needle is crescent-shaped as a whole, and a tail thread connection hole is provided at the tail of the surgical suture needle; The inner wall of the tail wire connection hole is provided with a circle of semi-circular protrusions; the coupling portion is inserted into the tail wire connection hole, and the semicircular card slot combined with the semi-circular protrusion, connect the double-stranded phoenix tail thread and the surgical stitch together; the double-stranded phoenix tail thread is used for bundling the fracture wounds that require double-strand sutures.

PCT Publication No. WO2019/045303A1 “Coupling Structure Of Multifilament Polydioxanone Suture And Needle Tube” discloses a suture that is inserted into the stomach and formed of polydioxanone to be absorbed into the body; The suture is coupled and includes a needle tube inserted into the body and a sponge for fixing the acupuncture tube and suture, the suture is coupled to and connected to the groove of the needle tube, and the needle tube and the suture are inserted and fixed in the sponge, and the suture is, a multifilament polydioxanone suture and acupuncture tube including a first suture coupled to the groove of the needle tube to form a ring and a plurality of second sutures connected to the first suture in a folded shape over the first suture combination structure.

U.S. Patent Publication No. 20180221021 “Braided Suture With Filament Containing A Medicant” by H. Scalzo, L. B. Kriksunov, R. J. Tannhauser, E. R. Skula, discloses a braided suture made from filament bundles, formed using at least two different filamentary materials, at least a first variety of filaments in a major portion, and a second variety of filaments in a minor portion incorporating a biomedically useful agent.

Despite recent advances, there remains an unmet need in the art to provide medical devices such as sutures with medicants while maintaining their mechanical strength and long-term integrity.

### SUMMARY

Presented is an implantable medical device, such as a surgical suture, attached to a needle for insertion into the tissue and passing the suture though the tissue, with a beneficial filament comprising a releasable medicant, said beneficial filament positioned alongside said suture and attached to the same needle or attached to the suture proximal to the needle.

The present invention is directed to suturing systems comprising a needle; an elongated flexible suture having a connecting end attached to said needle and an opposing free end; and at least one elongated external beneficial filament that is attached to said needle or attached to said suture at the connecting end. The beneficial filament has a smaller cross-sectional area and lower mechanical strength than the suture and has a medicant. The suture can be non-absorbable, while the beneficial filament is absorbable or soluble after installation into a tissue of a mammal. In another alternative, the suture can be absorbable while said beneficial filament is absorbable or soluble after installation into a tissue of a mammal. The beneficial filament can contain at least 20% by weight of the medicant. The suture can be barbed or knotless suture, while the beneficial filament is not barbed or knotless. The suture can be a braided filament construct, while said beneficial filament is a monofilament. The beneficial filament can be shorter than the suture.

In one embodiment, the suture comprises an indent that is configured to accept at least a portion of said beneficial filament. The beneficial filament can be attached to the needle and not be attached to the suture along the suture length. Alternatively, the beneficial filament can be secured to the suture along the suture length, either at every point of the suture or intermittently.

The beneficial filament can swellable in the cross section after installation into a tissue of a mammal by at least 25% within 60 minutes. Preferably, the mechanical properties of the suturing system are within 5% of the mechanical properties of the suture without the beneficial filament during and post-installation. For example, the suturing system can have a BSR (Breaking Strength Retention) that is substantially the same as BSR of said suture without the beneficial filament 1 week post-installation. The beneficial filament can be configured to fully dissolve or fully resorb within about 168 hours after installation into a tissue of a mammal. Alternatively, the beneficial filament can be configured to release at least 10% of the medicant within 24 hours after installation into a tissue of a mammal. The medicant can contain chlorhexidine, polyhexamethylene biguanide, octenidine, silver particles, silver salts, triclosan, and combinations thereof. The beneficial filament can be sterilized separately and differently from the suture and/or needle or can be sterilized separately and in the same manner as the suture and/or needle.

The present invention is also directed to methods of suturing a mammal tissue by passing the suturing systems described herein through tissue for purposes of approximating the tissue areas and establishing the tissue support with the suture, pulling the beneficial filament alongside the suture though the tissue, optionally securing the suturing system with knots, leaving the beneficial filament in the tissue alongside the suture and allowing the medicant to be released into the tissue.

The present invention is also directed to kits for assembling the suturing systems described herein, prior to implantation, having an elongated flexible suture attached to a needle, and, stored in a detached and separate form, at least one elongated external beneficial filament, that is configured for attaching to said needle or to said suture at the connecting end. The external beneficial filament can be subjected to a sterilization treatment separately from said suture attached to the needle.

The present invention is also directed to methods of making the suturing systems described here by attaching the connecting end of the suture to a needle and attaching a beneficial filament to said needle or to said suture at the connecting end.

### BRIEF DESCRIPTION OF THE DRAWINGS

The present disclosure is susceptible to various modifications and alternative forms, specific exemplary implementations thereof have been shown in the drawings and are herein described in detail. It should be understood, however, that the description herein of specific exemplary implementations is not intended to limit the disclosure to the particular forms disclosed herein.

This disclosure is to cover all modifications and equivalents as defined by the appended claims. It should also be understood that the drawings are not necessarily to scale, emphasis instead being placed upon clearly illustrating principles of exemplary embodiments of the present invention. Moreover, certain dimensions may be exaggerated to help visually convey such principles. Further where considered appropriate, reference numerals may be repeated among the drawings to indicate corresponding or analogous elements. Moreover, two or more blocks or elements depicted as distinct or separate in the drawings may be combined into a single functional block or element. Similarly, a single block or element illustrated in the drawings may be implemented as multiple steps or by multiple elements in cooperation.

The forms disclosed herein are illustrated by way of example, and not by way of limitation, in the figures of the accompanying drawings and in which like reference numerals refer to similar elements and in which:

FIGS. 1a-1e present schematic representations of a side cross-sectional view of embodiments of the suture system according to the present invention.

FIG. 2 presents a schematic representation of a side cross-sectional view of an embodiment of the suture system according to the present invention.

FIG. 3 presents a schematic representation of a side cross-sectional view of an embodiment of the suture system according to the present invention.

FIG. 4 presents a schematic representation of a side cross-sectional view of an embodiment of the suture system according to the present invention.

FIG. 5 presents a schematic representation of a side cross-sectional view of an embodiment of the suture system according to the present invention.

FIG. 6 presents a schematic representation of a side cross-sectional view of an embodiment of the suture system according to the present invention.

FIG. 7 presents a schematic representation of a side cross-sectional view of an embodiment of the suture system according to the present invention.

FIG. 8 presents a schematic representation of a side cross-sectional view of an embodiment of the suture system according to the present invention.

FIG. 9 presents a schematic representation of a side cross-sectional view of an embodiment of the suture system according to the present invention.

FIG. 10 presents a schematic representation of a side cross-sectional view of an embodiment of the suture system according to the present invention.

FIG. 11 presents a schematic representation of a side cross-sectional view of an embodiment of the suture system according to the present invention.

FIGS. 12a, 12b and 12c present schematic representations of a side view of embodiments of the suture system according to the present invention.

FIGS. 13a and 13b present schematic representations of a perspective view of embodiments of the suture system according to the present invention.

FIGS. 14a-14f present schematic representations of axial cross-sectional view of embodiments of the suture system according to the present invention.

FIGS. 15a-15d present schematic representations of axial cross-sectional view of embodiments of the suture system according to the present invention.

FIGS. 16a-16f present schematic representations of axial cross-sectional view of embodiments of the suture system according to the present invention.

FIGS. 17a-17c present schematic representations of axial cross-sectional view of embodiments of the suture system according to the present invention, shown installed in the tissue of a mammal.

### DETAILED DESCRIPTION

Briefly, the inventive suture system advantageously enables delivery of a medicant, or an additional or increased amount of a medicant, such as anti-bacterial, and/or healing promoting, and/or swellable medicant to the sutured wound or tissue, using any surgical suture (monofilament, braid, absorbable, non-absorbable, etc.), without changing the material, coatings, and properties of the main surgical suture. There delivery is provided by having at least one beneficial filament containing an additional medicant and/or swellable material, and/or sensing element, and having a smaller cross-section relative to the main surgical suture, and further having weaker mechanical properties (such as tensile strength, breaking strength, BSR, etc.) and having a faster dissolution or absorption profile relative to the main surgical suture. The beneficial filament is attached to the same suturing needle as the main suture or is attached to the main suture proximal to the needle. The beneficial filament is arranged alongside the main surgical suture, and is installed into the tissue alongside the main surgical suture. Thus, the overall strength of wound closure and suture knots has a baseline level that is defined by the surgical suture, while the beneficial filament, installed alongside the surgical suture into the tissue, provides additional medicant, such as an antimicrobial medicant, or wound healing agent, and/or swelling action useful to plug the suture holes (such as for instance for high pressure vessel closure). The perceived handling of the surgical suture is not affected because the primary surgical suture remains the same. In some embodiments, the knot strength is increased due to the presence of the beneficial filament.

### Construct

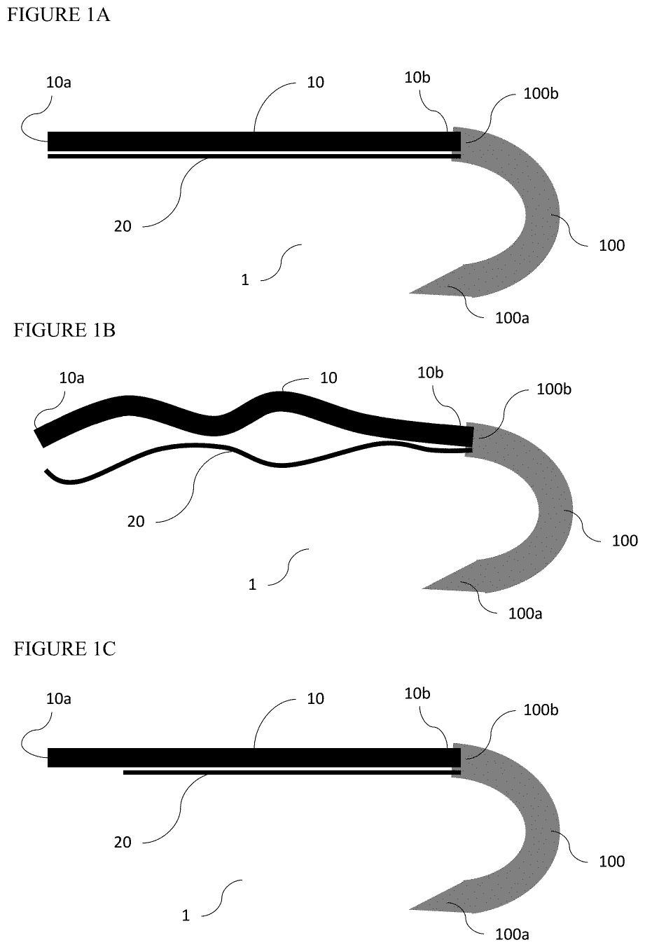

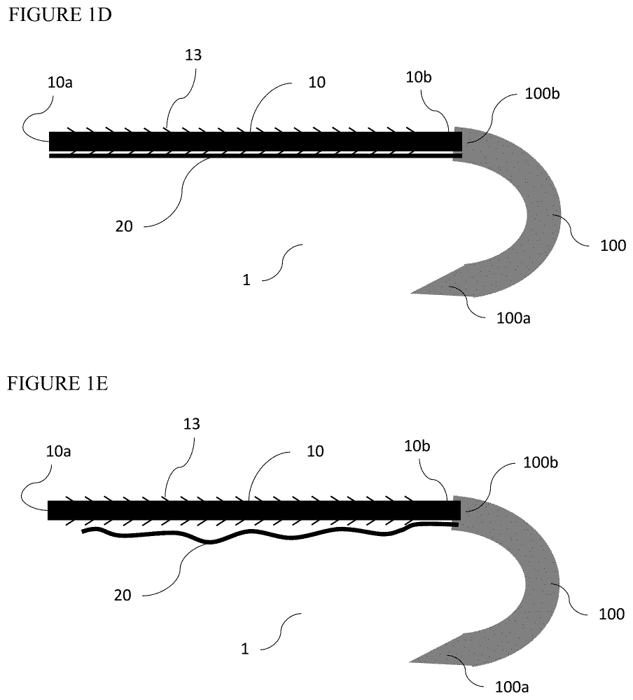

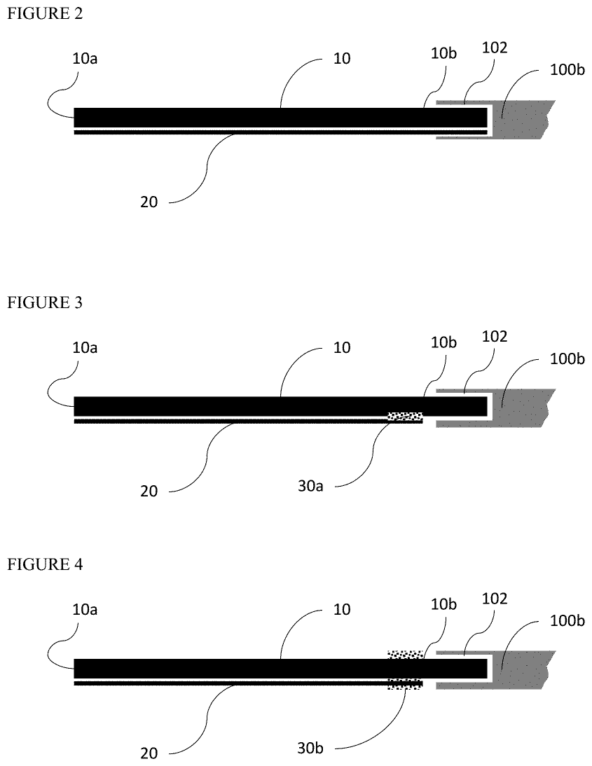

Referring to FIGS. 1A through 1E, showing a schematic side cross-sectional view, in one embodiment, the inventive suture system 1 comprises an elongated flexible string, filament, yarn, bundle of filaments, braid, comprising suture 10 having connecting end 10b and opposing free end 10a, with suture 10 connecting end 10b connected to suture needle 100 at a needle connecting end 100b. Needle 100 comprises an elongated straight or curved member having a sharpened end 100a opposite the needle connecting end 100b. As shown, and further referring to FIG. 2, suture 10 connecting end 10b is typically inserted into an opening 102 in needle connecting end 100b. Suture 10 connecting end 10b can be fixated in the opening 102 by swaging needle 100, using adhesives, etc. Free end 10a can optionally have a stop or tab (not shown) that fixates free end 10a in tissue.

Suture 10 can be any suture known in the art, including monofilament or multifilament or braided suture, absorbable or non-absorbable, or partially absorbable suture, made of any biocompatible material, including synthetic or natural materials, and combinations thereof. In some embodiments, polymeric materials of suture 10 comprise bio-resorbable polyester or non-bio-resorbable polypropylene. Particularly preferred as suture 10 are sutures approved for use in surgical procedures, for example sutures sold by Ethicon, Inc. under various brand names, such as VICRYL® (polyglactin 910) Suture, Coated VICRYL® (polyglactin 910) Suture; Coated VICRYL® Plus Antibacterial (polyglactin 910) Suture; VICRYL RAPIDE™ (polyglactin 910) Suture; MONOCRYL® (poliglecaprone 25) Suture; PDS® (polydioxanone) Suture; PDS® Plus Antibacterial (polydioxanone) Suture; ETHILON® Nylon Suture; ETHIBOND® Polyester Suture; MERSILENE® Polyester Fiber Suture; PRONOVA® Poly (Hexafluoropropylene—VDF) Suture; PROLENE® Polypropylene Suture; NUROLON® Nylon Suture, etc. Suture 10 can also optionally be a barbed or knotless suture, such as STRATAFIX™ Spiral Knotless Tissue Control Device; STRATAFIX™ Symmetric Knotless Tissue Control Device, or similar. Suture 10 can be an antimicrobial suture.

Needle 100 can be any surgical suture needle known in the art, including straight or curved needles, needles with various geometries of the needle body and tip or sharp end, needles coated or uncoated with lubricious coatings, and needles made of metal, such as steel, tungsten alloys, etc., or needles made of polymeric materials. Example include suture needles sold by Ethicon, Inc. under various brand names, such as EVERPOINT® Cardiovascular Needle; ETHALLOY® needle alloy based needles; HEMO-SEAL™ Needle Suture; ETHIGUARD® Blunt Point Needle; MULTIPLASS® Needles; VISI-BLACK™ Surgical Needle. Needles such as taper point, taper cut, cutting edge needles, etc., and similar, can be utilized. In one embodiment, the inventive suture system can be double armed (not shown) whereby a second needle (not shown) is attached at the free end 10a.

As shown in FIGS. 1 and 2, the inventive suture system 1 has at least one external beneficial string or yarn or filament 20 that is positioned alongside suture 10 and attached to the same needle as main suture 10. In the embodiments shown, filament 20 is fixated in opening 102 together with suture 100 by swaging needle 100, using adhesives, etc.

Referring to FIG. 1A, filament 20 is arranged alongside suture 10 and has the same length as suture 10. FIG. 1B, shows filament 20 attached to needle 100 and positioned in a free form and unattached alongside suture 10 and having the same length as suture 10. In some embodiments, as shown in FIG. 1C, filament 20 is shorter than suture 10, such as having length shorter by 5, 10, 15, 20, 30, 50%, each as a percentage of the length of suture 10. In some embodiments, filament 20 is shorter than suture 10 by 5, 10, 15, 20, 30, 50, 100, each as measured in mm.

Referring to FIGS. 1D and 1E, in some embodiments, suture 10 is a barbed suture having barbs 13, while filament 20 has no barbs in any embodiments. When suture 10 is a barbed suture, filament 20 can be attached at some points to suture 10 along suture 10 or not attached anywhere besides attachment in the proximity to connecting end 10b, as shown in FIG. 1E.

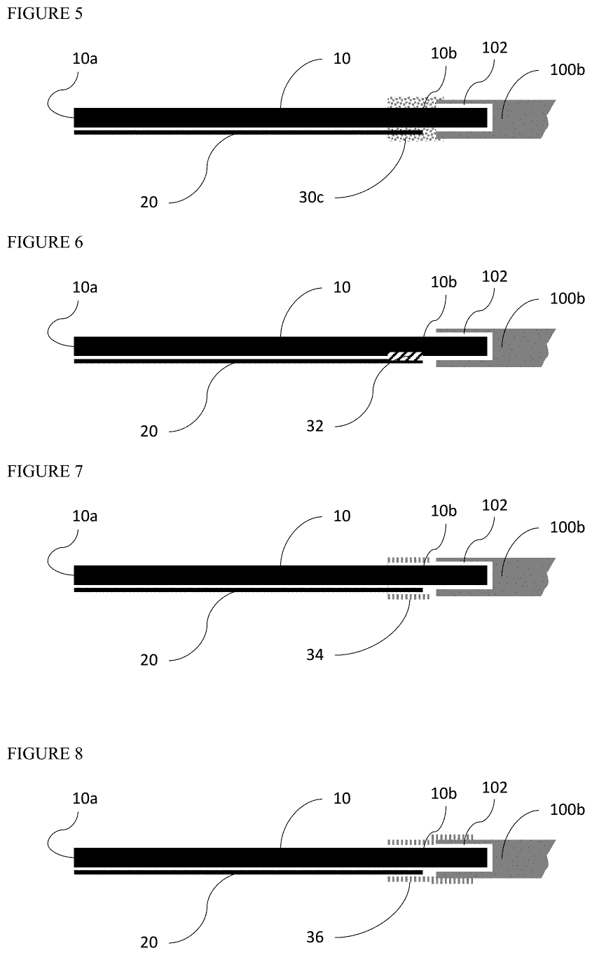

In an embodiment shown in FIG. 3, filament 20 is not attached to needle 100 through insertion into opening 102 as seen in FIGS. 1 and 2. In this embodiment, filament 20 is secured to suture 10 connecting end 10b by adhesive 30a disposed between suture 10 connecting end 10b and filament 20. As shown in FIG. 4, adhesive 30b can also extend around connecting end 10b and filament 20. As shown in FIG. 5, adhesive 30c can also extend around connecting end 10b and filament 20 and also contact needle connecting end 100b.

In an embodiment shown in FIG. 6, filament 20 is secured to suture 10 connecting end 10b by spot welding or melting, showing melted or fused area 32.

In an embodiment shown in FIG. 7, beneficial filament 20 is secured to suture 10 connecting end 10b by a sleeve 34, such as heat-shrinkable or adhesive sleeve. In an embodiment shown in FIG. 8, filament 20 is secured to suture 10 connecting end 10b by a sleeve 36, such as heat-shrinkable or adhesive sleeve 36 that is also overlying needle connecting end 100b.

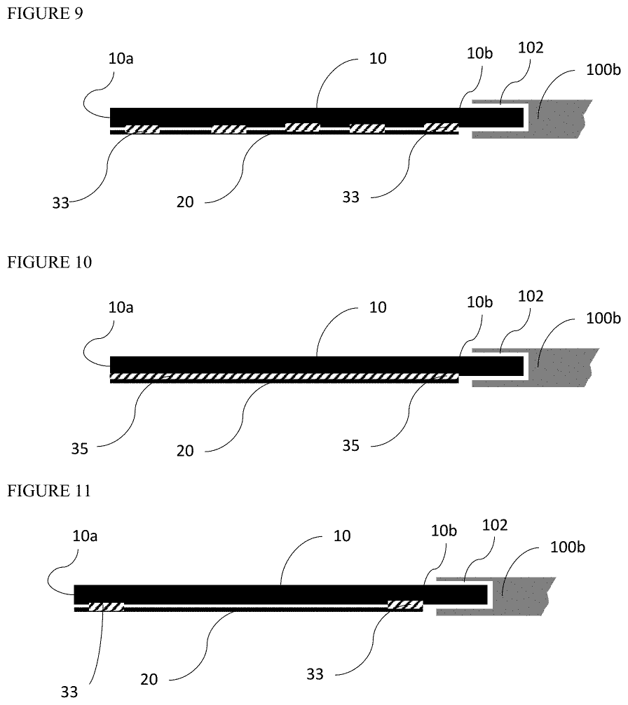

In the embodiments shown in FIGS. 1-8, filament 20 is positioned alongside suture 10 and is secured to suture 10 connecting end 10b either by attaching to suture 10, to needle 100, or to both. In further embodiments, beneficial filament 20 is also secured to suture 10 along suture 10 length, along at least 30% of length of suture 10, such as along 30, 50, 75, 100% of suture 10 length, either at every point of suture 10 length or intermittently. Referring to FIG. 9, filament 20 is secured to suture 10 along the whole length of suture 10 length by intermittent spot welds or adhesive spots 33. Intermittent spot welds or adhesive spots 33 can be 0.5-15 mm long, such as about 1, 2, 3, 5 mm long, and separated by gaps of about 2 to 50 mm, such as 2, 3, 5, 10, 20, 30 mm gaps. Referring to FIG. 10, filament 20 is secured to suture 10 along the whole length of suture 10 length by continuous welds or adhesive 35. Referring to FIG. 11, beneficial filament 20 is secured to suture 10 at the connecting end 10b and also secured at the opposing free end 10a by intermittent spot welds or adhesive spots 33.

One further technique that can be utilized to secure beneficial filament 20 to suture 10 is performed by exposing the beneficial filament 20 arranged alongside suture 10 to a liquid such as water, aqueous solution, pure solvent (such as ethanol), solution in a non-aqueous solvent, or to a vapor (such as water vapor, ethanol vapor, or similar), by dipping, spraying, vapor cabinet exposure, or similar. While pressing the beneficial filament 20 to suture 10, exposure to water, solvents, solutions, or vapor, results in beneficial filament 20 sticking to suture 10. Subsequent drying removes solvent or moisture, while leaving beneficial filament 20 attached to suture 10 in the areas where they were pressed together. In such embodiments, presence of a liquid softens and partially transiently solubilizes beneficial filament 20, making it sticky and adhesive. In another embodiment, adhesion between suture 10 and beneficial filament 20 is achieved by use of a solution of a soluble absorbable material, such as polyethylene glycol (PEG), polysaccharide, such as CMC, polyester, or similar. After evaporation of the solvent form such solution during drying, the remaining absorbable material serves to attach beneficial filament 20 to suture 10.

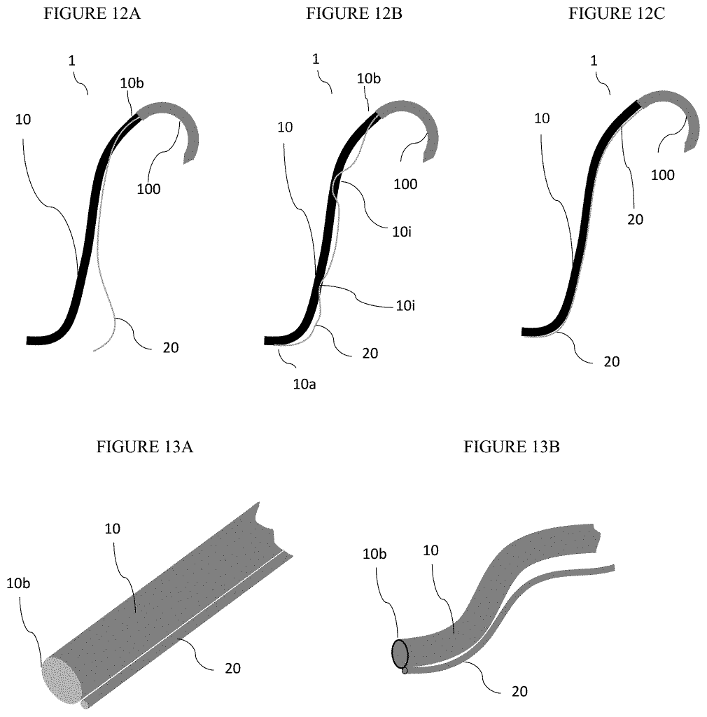

Embodiments of the inventive suture system 1 are further schematically shown in FIG. 12 in a side view. FIG. 12A shows beneficial filament 20 secured to suture 10 proximal to connecting end 10b only. FIG. 12B shows beneficial filament 20 secured to suture 10 at connecting end 10b, as well as at intermediate spots 10i along suture 10 length, and at the free end 10a. FIG. 12C shows beneficial filament 20 secured to suture 10 along the whole length of suture 10.

FIGS. 13Aand 13B schematically show a perspective view of positioning of beneficial filament 20 along suture 10, with FIG. 13A showing an embodiment with a side-by-side arrangement, with means of attachment or securement of beneficial filament 20 to suture 10 not only in the area of suture 10 connecting end 10b but along the whole length, attaching via adhesive, welding, sleeves, etc. (not shown in FIG. 13A). FIG. 13B shows an embodiment with attachment of beneficial filament 20 and suture 10 only in the area of suture 10 connecting end 10b.

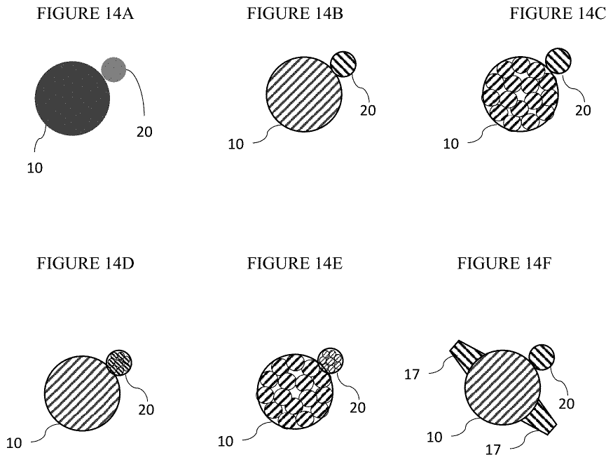

FIG. 14 shows schematic axial cross-sectional views showing arrangement of beneficial filament 20 alongside suture 10. FIG. 14A shows general view, while FIG. 14B shows an embodiment where both beneficial filament 20 and suture 10 comprise monofilaments, i.e. are made of a single string of material. FIG. 14C shows an embodiment where beneficial filament 20 comprises monofilament, while suture 10 comprises a braid, i.e. a bundle made of a plurality of yarns woven together, such bundle braided together to form a stable inter-braided construct or structure. FIG. 14D shows an embodiment where beneficial filament 20 comprises a braid, i.e. is made of a plurality of braided together yarns, while suture 10 comprises a monofilament. FIG. 14E shows an embodiment where both beneficial filament 20 and suture 10 comprise a braid. FIG. 14F shows an embodiment where suture 10 comprises a barbed suture having barbs 17 protruding outwards, similar to the STRATAFIX™ Symmetric Knotless Tissue Control Device, available form Ethicon, Inc. Advantageously, beneficial filament 20 is arranged between barbs 17 so as to not to interfere with barbs 17.

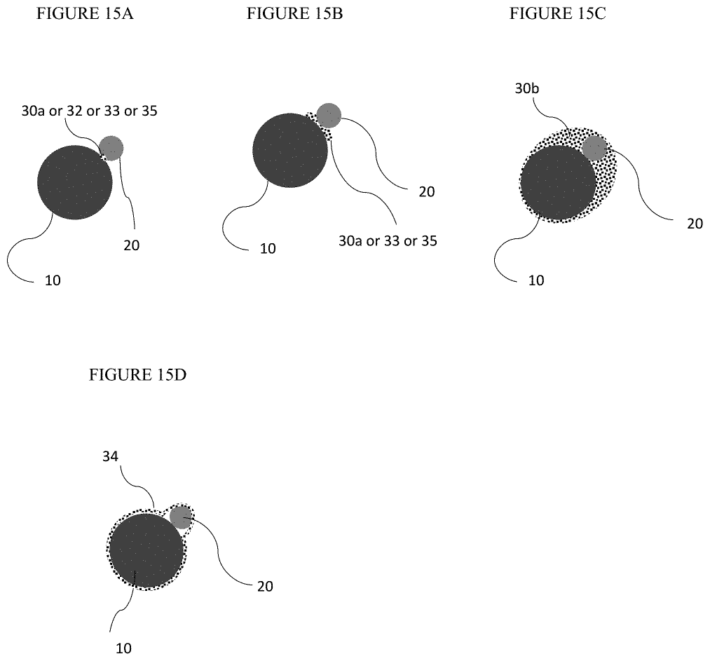

FIG. 15 shows schematic axial cross-sectional views showing arrangement of beneficial filament 20 alongside suture 10. FIG. 15A shows adhesive 30a or melted or fused area 32 or intermittent spot welds or adhesive spots 33 or continuous welds or adhesive 35 disposed between beneficial filament 20 and suture 10 and securing beneficial filament 20 to suture 10. FIG. 15B shows adhesive 30a or 33 or 35 disposed between beneficial filament 20 and suture 10 and securing beneficial filament 20 to suture 10. FIG. 15C shows adhesive 30b extending around beneficial filament 20 and suture 10 and securing beneficial filament 20 to suture 10. FIG. 15D shows sleeve 34, such as heat-shrinkable or adhesive sleeve, extending around beneficial filament 20 and suture 10 and securing beneficial filament 20 to suture 10.

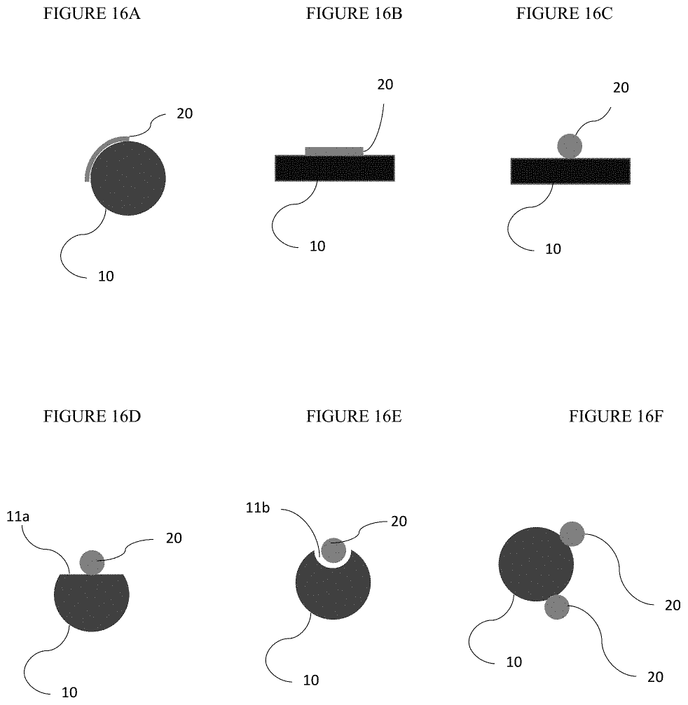

Advantageously, any cross-sectional shape of suture 10 and beneficial filament 20 can be utilized. While preferred embodiments shown are utilizing generally round or circular cross-sectional shapes, alternative cross-sections of any suitable geometric form can be used. FIG. 16 shows schematic axial cross-sectional views with some exemplary geometries, showing arrangement of beneficial filament 20 alongside suture 10. FIG. 16A shows beneficial filament 20 of arcuate or hollow semicircle shape fitting alongside generally circular suture 10, resulting in a compact construct for ease of installation into tissue. FIG. 16B shows beneficial filament 20 and suture 10 both of ribbon shape positioned alongside each other resulting in a compact construct for ease of installation into tissue. FIG. 16C shows a circular beneficial filament 20 alongside suture 10 of ribbon shape. FIG. 16D shows circular suture 10 having indent or flat 11a, whereby circular beneficial filament 20 is positioned alongside suture 10 in the indent 11 resulting in a compact construct for ease of installation into tissue. FIG. 16E shows circular suture 10 having a semicircular cutout or hollow 11b, shaped for accommodating at least a portion of beneficial filament 20, whereby circular beneficial filament 20 is positioned alongside suture 10 in the hollow 11b resulting in a compact construct for ease of installation into tissue. FIG. 16F shows two beneficial filaments 20 fitting alongside generally circular suture 10. While at least one beneficial filament 20 is shown in most embodiments, 2, 3, 4 of beneficial filaments 20 or more can be employed, with most preferred systems comprising one or two beneficial filaments 20 alongside suture 10. Two different beneficial filaments 20 can contain different types of medicant.

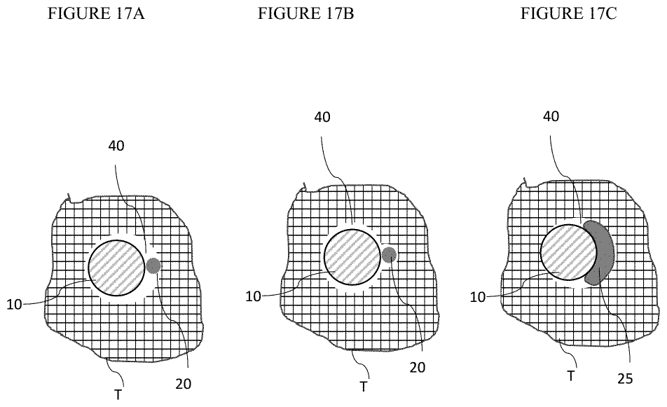

FIG. 17 shows schematic axial cross-sectional views of suture system installed in tissue T. FIGS. 17A and 17B show beneficial filament 20 alongside generally circular suture 10 within tissue T opening 40. Tissue opening 40 is formed as needle 100 (not shown) pierces tissue T during suture 10 installation.

FIG. 17C shows swellable beneficial filament 25 alongside generally circular suture 10 within tissue T opening 40, after installation and swelling of swellable beneficial filament 25, with expansion of swellable beneficial filament 25 filling space within tissue opening 40 and at least partially plugging tissue opening 40, thus preventing or decreasing bleeding through tissue opening 40.

### Construct Properties

In the preferred embodiments, suture 10 comprises any commercially available suture that is approved for use by relevant regulatory authorities. Thus, suture 10 maintains desirable mechanical, handling, knot strength, and absorbability properties, corresponding to approved and marketed sutures.

Suture 10 itself can have antimicrobial or any other medically beneficial properties (such as e.g. agents to assist or stimulate wound healing) or swellable coatings and/or antimicrobial or swellable components incorporated into suture 10 or alternatively can have no antimicrobial or swellable coatings and no antimicrobial or swellable components incorporated into suture 10. Advantageously, the inventive suture system 1 provides additional antimicrobial activity or swellability properties compared to commercially available and approved sutures, due to the presence of beneficial filament 20.

In one embodiment, beneficial filament has a minor impact or even no effect on mechanical properties of the inventive suture system 1 during and post-installation, whereby the beneficial filament mechanical strength may be low due to smaller cross-section and weaker material of construction.

Dimensionally, beneficial filament 20 cross-sectional area is less than 25% relative to the cross-sectional area of suture 10. Alternatively, in case of circular or round cross-sectional shapes, the dimensional relationship can be expressed relative to the diameter of beneficial filament 20 as 50% or less of the diameter of suture 10, resulting in cross-sectional area of beneficial filament 20 being 25% or less relative to the cross-sectional area of suture 10. Most preferably, the diameter of beneficial filament 20 is 50, 40, 30, 20, 10, 5% of the diameter of a circular shaped suture 10. Alternatively, more generally, the cross-sectional area of beneficial filament 20 is 25, 20, 15, 10, 5, 4, 3, 2, 1% of cross-sectional area of suture 10. The same ratios of cross-sectional areas relationships can be applied for the non-round/non-circular cross-sectional geometries.

Mechanical properties, such as tensile strength, and/or elongation under load, and/or breaking strength, and/or knot strength and/or knot slide of inventive suture system comprising combination of suture 10 and at least one beneficial filament 20 are either identical or very close to the properties of suture 10 alone, prior to installation and after tissue exposure in vivo or in a tissue model simulating installation in tissue, such as in aqueous system with temperature/buffer simulating tissue environment. As outlined in Table 1, mechanical properties of the inventive suture system 1 in the Embodiment A1 are identical to suture 10 alone within error of measurement, prior to installation into tissue or a tissue model, measured after 24 hours in tissue or tissue model, measured after 48 hours in tissue or tissue model, and measured after 72 hours in tissue or tissue model. Mechanical properties of the inventive suture system 1 in the Embodiment B1 are within 5% of suture 10 alone prior to installation into the tissue or tissue model and measured after 24 hours in tissue or tissue model; measured after 48 and 72 hours in tissue or tissue model mechanical properties of the inventive suture system 1 are identical to suture 10 alone within error of measurement. Mechanical properties of the inventive suture system 1 in the Embodiment C1 are within 10% of suture 10 alone prior to installation into the tissue or tissue model and measured after 24, 48 hours in tissue or tissue model; measured after 72 hours in tissue or tissue model mechanical properties of the inventive suture system 1 are identical to suture 10 alone within error of measurement.

The mechanical properties of the suture system 1 are close to or identical to such properties of the suture 10 alone due to:
Beneficial filament 20 has a smaller cross-section than suture 10
Beneficial filament 20 has lower mechanical strength than suture 10
Beneficial filament 20 has faster dissolution and/or resorption in the tissue relative to suture 10

TABLE 1
Mechanical properties for various embodiments

| | Exposure to | | | |
|---|---|---|---|---|
| | tissue: timing | Embodiment A1 | Embodiment B1 | Embodiment C1 |
| Mechanical properties, such | Prior to | Identical to | Within 5% of | Within 10% |
| as tensile strength, and/or | installation | suture 10 alone | suture 10 alone | suture 10 alone |
| elongation under load, and/or | into the tissue | within error of | | |
| breaking strength, and/or | or tissue model | measurement | | |
| knot strength, and/or knot | Measured after | Identical to | Within 5% of | Within 10% |
| slide of inventive suture | 24 hours in | suture 10 alone | suture 10 alone | suture 10 alone |
| system comprising | tissue or tissue | within error of | | |
| combination of suture 10 and | model | measurement | | |
| at least one beneficial | Measured after | Identical to | Identical to | Within 10% |
| filament 20; prior to | 48 hours in | suture 10 alone | suture 10 alone | suture 10 alone |
| installation and after tissue | tissue or tissue | within error of | within error of | |
| exposure in vivo or in a | model | measurement | measurement | |
| tissue model simulating | Measured after | Identical to | Identical to | Identical to |
| installation in tissue, such as | 72 hours in | suture 10 alone | suture 10 alone | suture 10 alone |
| in aqueous system with | tissue or tissue | within error of | within error of | within error of |
| temperature/buffer | model | measurement | measurement | measurement |
| simulating tissue | | | | |
| environment | | | | |

### Materials

### Suture 10 Materials

Suture 10 materials can be any suture materials known in the art, particularly commercially available and approved suture materials, such as synthetic and natural biocompatible polymeric materials, which may be non-absorbable or absorbable. Examples of synthetic non-absorbable polymeric materials useful to manufacture non-absorbable sutures include polyesters, polyolefins, polyvinylidene fluorides and polyamides. Further examples of non-absorbable materials are polyethylene, polypropylene, nylon, and similar polymers. Examples of synthetic absorbable polymeric materials useful to manufacture absorbable sutures include polymers and copolymers made from lactones such as the lactides, glycolide, p-dioxanone, epsilon-caprolactone, and trimethylene carbonate. Suitable biocompatible, biodegradable polymers may be synthetic or natural polymers. Suitable synthetic biocompatible, biodegradable polymers include polymers selected from the group consisting of aliphatic polyesters, poly(amino acids), copoly(ether-esters), polyalkylene oxalates, tyrosine-derived polycarbonates, poly(iminocarbonates), polyorthoesters, polyoxaesters, polyamidoesters, polyoxaesters containing amine groups, poly(anhydrides), polyphosphazenes, and combinations thereof. For the purposes of this invention aliphatic polyesters include, but are not limited to, homopolymers and copolymers of lactide (which includes lactic acid, D-, L- and meso lactide), glycolide (including glycolic acid), .epsilon.-caprolactone, p-dioxanone (1,4-dioxan-2-one), trimethylene carbonate (1,3-dioxan-2-one), alkyl derivatives of trimethylene carbonate, and blends thereof. Polymers can be selected from poly(lactic acid) (PLA), polyglycolic acid (PGA), polycaprolactone (PCL), polylactide-glycolic acid (PLGA), polypropylene (PP), polyethylene (PE), polydioxanone (PDS), or combinations or copolymers of the monomers thereof. Suitable bioabsorbable, biocompatible elastomeric copolymers include but are not limited to copolymers of .epsilon.-caprolactone and glycolide (preferably having a mole ratio of .epsilon.-caprolactone to glycolide of from about 30:70 to about 70:30, preferably 35:65 to about 65:35, and more preferably 45:55 to 35:65); elastomeric copolymers of .epsilon.-caprolactone and lactide, including L-lactide, D-lactide blends thereof or lactic acid copolymers (preferably having a mole ratio of .epsilon.-caprolactone to lactide of from about 35:65 to about 65:35 and more preferably 45:55 to 30:70) elastomeric copolymers of p-dioxanone (1,4-dioxan-2-one) and lactide including L-lactide, D-lactide and lactic acid (preferably having a mole ratio of p-dioxanone to lactide of from about 40:60 to about 60:40); elastomeric copolymers of .epsilon.-caprolactone and p-dioxanone (preferably having a mole ratio of epsilon-caprolactone to p-dioxanone of from about 30:70 to about 70:30); elastomeric copolymers of p-dioxanone and trimethylene carbonate (preferably having a mole ratio of p-dioxanone to trimethylene carbonate of from about 30:70 to about 70:30); copolymers of trimethylene carbonate and glycolide (preferably having a mole ratio of trimethylene carbonate to glycolide of from about 30:70 to about 70:30); elastomeric copolymer of trimethylene carbonate and lactide including L-lactide, D-lactide, blends thereof or lactic acid copolymers (preferably having a mole ratio of trimethylene carbonate to lactide of from about 30:70 to about 70:30) and blends thereof. In one embodiment, the elastomeric copolymer is a copolymer of glycolide and .epsilon.-caprolactone. In another embodiment, the elastomeric copolymer is a copolymer of lactide and .epsilon.-caprolactone. Non-absorbable, i.e. biodurable suture materials can include nylon, polyethylene, polypropylene or copolymers of the monomers thereof. Suitable biodurable polymers include, but are not limited to polyurethane, polypropylene (PP), polyethylene (PE), polycarbonate, polyamides, such as nylon, polyvinylchloride (PVC), polymethyl-methacrylate (PMMA), polystyrene (PS), polyester, polyetheretherketone (PEEK), polytetrafluoroethylene (PTFE), polytrifluorochloroethylene (PTFCE), polyvinylfluoride (PVF), fluorinated ethylene propylene (FEP), polyacetal, polysulfone, silicones, and combinations thereof.

### Beneficial Filament 20 Materials

Beneficial filament 20 incorporates a quantity of medicant, and can contain up to 80% of medicant, such as 5, 10, 20, 30, 40, 50, 60, 70, 75, 80% of medicant by weight. Advantageously, such large quantity of medicant results in a mechanically weak filament that is faster/more rapidly soluble/absorbent relative to suture 10.

Preferably, medicant is compounded into the beneficial filament 20 material, wherein the beneficial filament 20 compromises a mixture of medicant and polymeric binder, and such mixture is extruded to form elongated beneficial filament 20.

Beneficial filament 20 is in one embodiment configured to rapidly release the medicant after installation into the tissue. As shown in Table 2 for embodiments D1, E1, F1, beneficial filament 20 is configured to release at least 5, 10, 20% of the medicant within 24 hours in tissue or tissue model; release at least 10, 25, 50% of the medicant within 48 hours in tissue or tissue model; release at least 20, 50, 90% of the medicant within 72 hours in tissue or tissue model.

TABLE 2
Medicant release

| | Exposure to | | | |
|---|---|---|---|---|
| | tissue | Embodiment D1 | Embodiment E1 | Embodiment F1 |
| Medicant release from the | After 24 hours | Release at least | Release at least | Release at least |
| beneficial filament after | in tissue or | 20% of the | 10% of the | 5% of the |
| tissue exposure in vivo or in | tissue model | medicant | medicant | medicant |
| a tissue model simulating | After 48 hours | Release at least | Release at least | Release at least |
| installation in tissue, such as | in tissue or | 50% of the | 25% of the | 10% of the |
| in aqueous system with | tissue model | medicant | medicant | medicant |
| temperature/buffer | After 72 hours | Release at least | Release at least | Release at least |
| simulating tissue | in tissue or | 90% of the | 50% of the | 20% of the |
| environment | tissue model | medicant | medicant | medicant |

In some embodiments, beneficial filament is configured to substantially fully dissolve and/or resorb after tissue exposure in vivo or in a tissue model simulating installation in tissue, such as in aqueous system with temperature/buffer simulating tissue environment, within 24 hours, after 48 hours, after 72 hours, after 96 hours, after 120 hours, or after 168 hours.

Beneficial filament 20 comprises at least one medicant and at least one absorbable and/or soluble filament-forming material that is mixed with the medicant, or binds the medicant, or generally contains or holds the medicant within the filament-forming material matrix and enables forming an elongated string via melt extrusion, solvent extrusion, casting, pulling, or any available technique.

Preferably, the filament-forming material of the beneficial filament 20 is a natural or synthetic polymer or mix of polymers. Suitable natural polymers include, but are not limited to collagen, gelatin, elastin, polymerized hyaluronic acid, cellulose, oxidized cellulose, or any naturally derived polymer or combinations thereof with natural or synthetic polymers. Suitable synthetic polymers include, but are not limited to, biocompatible polymers selected from the group consisting of polyacrylamide, polyvinylpyrrolidone, polyvinyl alcohol, polyvinylmethylether, polyethylene oxide, polyethylene glycol, and combinations of any of these.

Release of the antimicrobial agent or medicant from beneficial filament 20 can be instead of or in addition to any antimicrobial agents/medicant released by the suture 10 itself.

### Medicants

Suitable antimicrobial agents can be those which can be homogenously distributed throughout the polymer matrix of the abradable coating. For example, the antimicrobial agent can be any medicant having antibiotic or antimicrobial function, including chlorhexidine, polyhexamethylene biguanide (PHMB), octenidine, silver particles, silver salts, triclosan, and combinations thereof. Suitable antimicrobial agents may be selected from, but are not limited to, halogenated hydroxyl ethers, acyloxydiphenyl ethers, or combinations thereof. In particular, the antimicrobial agent may be a halogenated 2-hydroxy diphenyl ether and/or a halogenated 2-acyloxy diphenyl ether, esters of acetic acid, chloroacetic acid, methyl or dimethyl carbamic acid, benzoic acid, chlorobenzoic acid, methylsulfonic acid and chloromethylsulfonic acid are particularly suitable. Some particularly advantageous antimicrobial agents are 2,4,4′-trichloro-2′-hydroxydiphenyl ether, commonly referred to as triclosan, chlorhexidine gluconate, and combinations thereof.

In one form, material having high affinity for the antimicrobial agent or particles of such material can be distributed throughout the polymer matrix of beneficial filament 20. For example, polycaprolactone and co-polymers based on polycaprolactone have a high affinity for triclosan. Thus, particles of (co)polymers of polycaprolactone or polycaprolactone itself can be included in the beneficial filament 20 when triclosan is selected as the antimicrobial agent. Beneficial filament 20 can comprise a material having a high solubility of biomedically useful agent, for example at least about 30 wt % polycaprolactone and the biomedically useful agent is triclosan. Triclosan is known to vaporize under relatively mild temperature and/or low pressure conditions and will be preferentially absorbed into the polycaprolactone-containing polymer.

In one form, particles such as silver particles having sizes from about 0.1 micron to about 300 microns, such as about 10, about 50, about 100, or even about 150 microns, are incorporated into beneficial filament 20 in concentrations from about 1% to about 75% by volume, such as from about 2, 3, 5, 10, 15, 20, 30, or 50% by volume. In some forms, silver salts can be used, such as carbonates, lactides, nitrates, or similar.

In addition to the antimicrobial agents described herein, beneficial filament 20 medicant can be a biocide, a disinfectant and/or an antiseptic, including but not limited to alcohols such as ethanol and isopropanol; aldehydes such as glutaraldehyde and formaldehyde; anilides such as triclorocarbanilide; biguanides such as chlorhexidine; chlorine-releasing agents such as sodium hypochlorite, chlorine dioxide and acidified sodium chlorite; iodine-releasing agents such as povidone-iodine and poloxamer-iodine; metals such as silver nitrate, silver sulfadiazine, other silver agents, copper-8-quinolate and bismuth thiols; peroxygen compounds such as hydrogen peroxide and peracetic acid; phenols; quaternary ammonium compounds such as benzalkonium chloride, cetrimide and ionenes-polyquaternary ammonium compounds.

Beneficial filament 20 may incorporate medicants such as antibiotics, including but not limited to penicillins such as amoxicillin, oxacillin and piperacillin; cephalosporins parenteral such as cefazolin, cefadroxil, cefoxitin, cefprozil, cefotaxime and cefdinir; monobactams such as aztreonam; beta-lactamase inhibitors such as clavulanic acid sulbactam; glycopeptide such as vancomycin; polymixin; quinolones such as nalidixic acid, ciprofloxacin and levaquin; metranidazole; novobiocin; actinomycin; rifampin; aminoglycosides such as neomycin and gentamicin; tetracyclines such as doxycycline; chloramphenicol; macrolide such as erythromycin; clindamycin; sulfonamide such as sulfadiazine; trimethoprim; topical antibiotics; bacitracin; gramicidin; mupirocin; and/or fusidic acid.

In some embodiments, the medicant further comprises antibodies, growth factors, cytokines , chemokines, wound healing enhancing agents, therapeutic peptides, and combinations thereof.

One beneficial filament 20 can comprise two or more different medicants incorporated therein. When two or more different beneficial filaments 20 are used, each can incorporate the same medicant(s) or each can contain different types of medicant. Thus a first beneficial filament 20 can incorporate a first medicant, e.g. chlorhexidine gluconeate or silver particles, and a second beneficial filament 20 can incorporate a second medicant, e.g. polyhexamethylene biguanide or silver salt.

### Sterilization and Assembly

In one embodiment, the inventive suturing system 1 is assembled and then sterilized and packaged or packaged and then sterilized. In this embodiment, sterilization of beneficial filament 20 secured to suture 10/needle 100 is performed together, by the same sterilization technique (e.g. Ethylene Oxide based, Gamma irradiation based, e-beam irradiation based, thermal based, etc.).

In an alternative embodiment, beneficial filament 20 is supplied unattached, but as a part of a kit with suture 10 and needle 100. In this case, beneficial filament 20 is secured to suture 10 and/or needle 100 forming suturing system 1 immediately before use on a patient, that is within 1-120 minutes prior to use, such as within 1-30 minutes prior to use. In this embodiment, sterilization of beneficial filament 20 unattached to suture 10/needle 100 can be performed at the same time, by the same sterilization technique (e.g. Ethylene Oxide based, Gamma irradiation based, e-beam irradiation based, thermal based, etc.), or separately, by different sterilization techniques. Advantageously, sterilizing beneficial filament 20 by a suitable sterilization technique that does not negatively affect the medicant while sterilizing suture 10/needle 100 by another suitable sterilization technique, can be performed separately and thus sterilized beneficial filament 20 and suture 10/needle 100 can be provided unattached as a part of sterile kit. In this embodiment, the healthcare professional performs attachment of beneficial filament 20 to suture 10/needle 100 in the surgical suite by using suitable sterile adhesive, heat-shrinkable sleeve, or thermal treatment to bond beneficial filament 20 to suture 10 and/or needle 100 thus forming suturing system 1 immediately before use on a patient.

### Signaling or Sensor

In some embodiments, beneficial filament 20 comprises a sensor, that is configured to indicate conditions in the tissue and/or in the wound. Particularly, conditions indicating undesirable processes such as infection, presence of infective agents, and biomarkers indicative of such are sensed and detected. In one embodiment, an indicator reflecting the presence of cancer-type cells can be incorporated. Direct detection of bacteria is contemplated, as well as biomarkers of inflammation and/or infection, such as antibodies, cytokines and chemokines and bacterial antigens and metabolites. Also contemplated is detection of physiological environment of the wound that can be indicative of infection, such as specific pH, oxygenation, temperature, etc. Alternatively, sensing and detection of normal conditions, indicating absence of inflammation and/or infection, is also contemplated.

Reporting and signaling of the conditions in the wound is accomplished by beneficial filament signaling, such as color changes. Alternatively, the beneficial filament is removed and analyzed. Alternatively, beneficial filament comprises electronic microchip and sensors and wired conduit or antenna for wired or wireless reporting. If electronic type sensors are utilized, the beneficial filament can synergistically interact with an already embedded sensor in a way to passively amplify a wireless signal from the already embedded senor, i.e., act to resonate or amplify or transmit the signal via an antenna effect.

In an alternative embodiment, beneficial filament 20 is configured to carry a signal from a sensor (not shown) installed in the tissue at the wound site, with beneficial filament 20 carrying an electronic signal, i.e. electric current, or serving as a transmission antenna for communicating information to a receiver/recorder outside of the tissue, by wired or wireless communication means. In this embodiment, with the sensor sensing the conditions in the tissue and or in the wound, the signal from the sensor is carried via beneficial filament 20 to an external receiver (not shown). Thus information on healing and or infection conditions in the wound is carried to outside and communicated to a health practitioner and or the patient, utilizing beneficial filament 20 to carry/transfer such information to the receiver external to the wound/patient, with beneficial filament 20 not necessarily generating the information or sensing the wound environment. In some embodiments, beneficial filament 20 is connected to the auxiliary in-tissue sensor after beneficial filament 20 installation in tissue, via wired or wireless link, and is connected to the external recorder, also via wired or wireless link, also after beneficial filament 20 installation in tissue.

### Swellable Beneficial Filament

In one embodiment, beneficial filament 20 is swellable upon exposure to moisture, body fluids, or tissue. Advantageously, upon use of suture system 1 for suturing, and installation of suture 10 with beneficial filament 20 in the tissue, beneficial filament swells and expands thus facilitating closing of needle puncture holed in tissue. Swelling and expansion of beneficial filament 20a is configured to occur within less than 60 minutes after installation in tissue, such as within 1, 2, 3, 5, 10, 30, 45 minutes. Swelling or expansion constitutes increase in the cross-section of the beneficial filament of at least 10%, such as 10, 20, 30, 50, 100, 200, 300, 500%.

FIG. 17C shows swellable beneficial filament 25 alongside generally circular suture 10 within tissue T opening 40, after installation and swelling of swellable beneficial filament 25, with expansion of swellable beneficial filament 25 filling space within tissue opening 40 and at least partially plugging tissue opening 40, thus preventing or decreasing bleeding through tissue opening 40.

Swelling materials are any biocompatible materials that increase volume or swell upon intake of water, including superabsorbent polymers, hydrogels, i.e. hydrophilic, three-dimensional networked material which are able to intake large amounts of water or biological fluids, gelatin, gelatin/polyvinyl pyrrolidone hydrogels, hydroxypropyl methylcellulose, poly(ethylene oxide), sodium alginate, etc.

The swellable beneficial filament 20 can further have releasable active medicants, such as antimicrobial agents coated or impregnated or compounded into the filament 20.

While the invention has been described herein with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims.

I/we claim:

1. A suturing system, comprising:
a) A needle;
b) An elongated flexible suture having a connecting end attached to said needle and an opposing free end; and
c) At least one elongated external beneficial filament that is attached to said needle or attached to said suture at the connecting end,
Wherein said beneficial filament has a smaller cross-sectional area and lower mechanical strength than the suture, and
Wherein said beneficial filament has a medicant.

2. The suturing system of claim 1, wherein said suture is non-absorbable and said beneficial filament is absorbable or soluble after installation into a tissue of a mammal.

3. The suturing system of claim 1, wherein said suture is absorbable and said beneficial filament is absorbable or soluble after installation into a tissue of a mammal.

4. The suturing system of claim 1, wherein said beneficial filament contains at least 20% by weight of the medicant.

5. The suturing system of claim 1, wherein said suture is barbed or knotless suture and said beneficial filament is not barbed or knotless.

6. The suturing system of claim 1, wherein said suture is a braided filament construct and said beneficial filament is a monofilament.

7. The suturing system of claim 1, wherein said beneficial filament is shorter than said suture.

8. The suturing system of claim 1, wherein said suture comprises an indent configured to accept at least a portion of said beneficial filament.

9. The suturing system of claim 1, wherein said beneficial filament is not attached to said suture along the suture length.

10. The suturing system of claim 1, wherein said beneficial filament is secured to said suture along the suture length, either at every point of the suture or intermittently.

11. The suturing system of claim 1, wherein said beneficial filament is swellable in the cross section after installation into a tissue of a mammal by at least 25% within 60 minutes.

12. The suturing system of claim 1, wherein mechanical properties of said suturing system are within 5% of the properties of said suture without the beneficial filament during and post-installation.

13. The suturing system of claim 1, wherein said suturing system has a BSR that is substantially the same as BSR of said suture without the beneficial filament 1 week post-installation.

14. The suturing system of claim 1, wherein said beneficial filament is configured to fully dissolve or fully resorb within about 168 hours after installation into a tissue of a mammal.

15. The suturing system of claim 1, wherein said beneficial filament is configured to release at least 10% of the medicant within 24 hours after installation into a tissue of a mammal.

16. The suturing system of claim 1, wherein said medicant comprises chlorhexidine, polyhexamethylene biguanide, octenidine, silver particles, silver salts, triclosan, and combinations thereof.

17. The suturing system of claim 1, wherein said beneficial filament is sterilized separately and differently from said suture and said needle or wherein said beneficial filament is sterilized separately and in the same manner as said suture and said needle.

18. A method of suturing a mammal tissue, comprising
Passing the suturing system of claim 1 through the tissue approximating the tissue areas and establishing the tissue support with the suture,
Pulling the beneficial filament alongside the suture though the tissue,
Optionally securing the suturing system with knots,
Leaving the beneficial filament in the tissue alongside the suture,
Allowing the medicant to be released into the tissue.

19. A kit for assembling the suturing system of claim 1, prior to implantation, comprising:
a) the elongated flexible suture attached to the needle, and
b) separate from the suture and needle at least one elongated external beneficial filament, configured for attaching to said needle or to said suture at the connecting end,
said external beneficial filament subjected to a sterilization treatment separately from said suture attached to the needle.

20. A method of making the suturing system of claim 1, comprising:
Attaching the connecting end of the suture to the needle, and
Attaching the beneficial filament to said needle or to said suture at the connecting end.
